(19) United States
(12) Patent Application Publication
(10) Pub. No.: US 2009/0263476 A1
(43) Pub. Date: Oct. 22, 2009

(54) Composition of Rapid Disintegrating Direct Compression Buccal Tablet

(76) Inventors: Christopher N. Jobdevairakkam, Plainsboro, NJ (US); Vikram Katragadda, Lawrenceville, NJ (US)

Correspondence Address: ST. ONGE STEWARD JOHNSTON & REENS, LLC, 986 BEDFORD STREET, STAMFORD, CT, 06905-5619, US

(21) Appl. No.: 12/423,552

(22) Filed: Apr. 14, 2009

Related U.S. Application Data
(60) Provisional application No. 61/045,305, filed on Apr. 16, 2008.

(51) Int. Cl. A61K 9/48; A61K 9/20; A61K 31/485

(52) U.S. Cl. 424/452; 424/465; 514/282

(57) ABSTRACT

A composition of a rapidly disintegrating buccal dosage form containing a drug, at least one non-effervescent base such as an alkali metal or alkaline earth metal oxide or hydroxide, and a disintegrant. The base regulates the pH gradient to deliver the drug to the buccal, sublingual or oral mucosal membranes at a desired rate of absorption. The composition is micronized for uniform distribution, and the drug is converted from ionized form to unionized form, without the use of an effervescent agent.

### CROSS-REFERENCE TO RELATED APPLICATIONS

The present application claims the benefit under 35 U.S.C. §119(e) of the Provisional Patent Application Ser. No. 61/045,305 filed Apr. 16, 2008.

### FIELD OF THE INVENTION

The present invention relates to a composition of a rapidly disintegrating buccal dosage form containing the medicament, such as a drug, at least one non-effervescent base such as an alkali metal oxide and/or alkaline earth metal oxide or hydroxide, and a disintegrant. The base used in the composition does not generate any gaseous products upon contact with saliva, and eventually it regulates the pH gradient useful to deliver the drug to the buccal or sublingual or oral mucosal membranes at a desired rate of absorption.

### BACKGROUND OF THE INVENTION

Opioids are among the most frequently used effective analgesics, as opioids are generally considered safe when administered per advised dosage levels. Opioids are typically administered through different routes, such as administration by buccal, sublingual, transmucosal, nasal, and intravenous delivery. Fentanyl is an opioid effectively administered on a microgram scale to manage pain and it is mostly prescribed for severe pain and breakthrough pain management. Breakthrough pain management is where a drug is expected to exhibit its analgesic activity within few minutes after the oral administration. It is very challenging to make fast delivery oral dosage forms capable of treating breakthrough pain management, as there always exists the risk of an uncontrolled burst release of the drug. The critical factors for fast delivery oral dosage forms are the uniformity of the drug within the dosage form and the regulated release rate of the drug. Typically, fast delivery oral dosage forms are soft tablets, hard tablets, or capsules which are generally administered through either the buccal or sublingual route.

U.S. Pat. No. 5,711,961 (Reiner) discloses a chewing gum dosage form to administer a drug, such as mebendazole, to patients. U.S. Pat. No. 5,298,256 (Flockhart) discloses an oral transmucosal delivery of a drug using a buccal patch. Tablets are designed specifically to deliver small amounts of a potent drug, which is almost immediately dissolved and absorbed in the buccal or sublingual cavity. U.S. Patent Application No. 2006/0210632 (Oury) discloses a sublingual coated tablet consisting of a compressed core of excipients and has the drug substance contained in the tablet coating.

U.S. Patent Application No. 2007/0031491 (Levine) discloses a bio-adhesive tablet suitable for buccal, vaginal, nasal, or rectal delivery, containing a bio-adhesive agent such as a water soluble polymer, which acts as a medium for transporting the drug from the matrix to the desired tissues and/or membranes.

U.S. Pat. No. 6,495,120 (McCoy) discloses a composition of an oral dosage form comprising at least one oral absorption enhancer such as hydroxypropyl-beta-cyclodextrin, benzalkonium chloride, benzethonium chloride, polysorbate 80, sodium lauryl sulfate, polyoxyethylene ethers of aliphatic alcohols, polyoxyethylene derivatives of fatty acid partial esters of sorbitol anhydride, or polyoxyalkylene block copolymers. U.S. Pat. No. 6,680,071 (Johnson) and U.S. Pat. No. 7,090,866 (Johnson) each disclose a pharmaceutical composition to administer fentanyl in a fast dispersing dosage form wherein the drug is freeze dried with the excipients and a water soluble or water dispersible carrier such as gelatin or Mannitol.

U.S. Pat. Nos. 7,087,215 and 6,849,263 to Modi et al disclose a method of enhancing oral absorption of drugs by delivering the drugs to the buccal mucosa in the form of a micelle administered through spray formulations or liquid formulations.

U.S. Pat. No. 6,761,910 and World patent application WO 2004/067004 (PCT/SE2004/000037) to Pettersson et al., disclose a rapidly acting pharmaceutical composition administered by sublingual or intranasal administration of a pharmaceutical agent using bio-adhesion or mucoadhesion agents that are essentially water insoluble and larger in particle size than that of the active agent. The bio-adhesion and mucoadhesion promoting agents are defined as cross linked polymers, acrylic polymers, cellulose derivatives, natural polymers and edible gums.

US Patent Application Publication No. 2005/0042281 (Singh) discloses compositions of chewing gum useful for delivering drugs by converting the drugs into unionized form. Also Singh discloses compositions of quickly dissolving tablets using effervescent buffers, whereby the adsorption of drug is enhanced by the basic pH and the gas generating agents.

U.S. Pat. No. 6,645,520 (Hsu) and U.S. Pat. No. 6,562,368 (Hsu) disclose the use of hydroxide releasing bases to deliver the NSAID active ingredients through a body surface. These patents utilize hydroxide releasing agents in the matrix to maintain the pH of the delivery system, such as a transdermal patch.

Compositions of buccal and sublingual tablets using at least one effervescent agent have also been disclosed and it is claimed that the effervescent agent (a gas generating agent) is responsible for the transportation of fentanyl across the buccal membranes. The pH of the tablet upon contact with saliva is claimed as substantially neutral, slightly higher than a pH of 7. Effervescent tablets are very much brittle and hygroscopic and thus it is very difficult to handle these tablets for use in large scale operations.

U.S. Pat. Nos. 6,680,071 and 7,090,866 to Johnson et al., have claimed a pharmaceutical composition to administer fentanyl in a fast dispersing dosage form administered in a sachet. The composition contained in a freeze-dried blend comprises the fentanyl citrate drug, a water soluble or water dispersible carrier such as gelatin, and Mannitol. Such a composition is claimed to disperse in the oral cavity in 1 to 60 seconds. It is important to note that the composition according to the '866 patent does not contain a disintegrating agent or a buffer. The matrix forming agents disclosed in the '071 and '866 patents are amino acid having 2 to 12 carbon groups, inorganic salts such as sodium phosphate sodium chloride or aluminum silicate, and sugars such as Mannitol, dextrose lactose galactose, trehalose and cyclodextrin. Though use of citric acid and glycine as part of the matrix, the significance of the buffer system was not claimed essential in the '866 and '071 patents.

Furthermore, the '866 and '071 patents do not teach the essential technology of administering fentanyl citrate by absorption to the oral cavities. It is inferred that the composition of the '866 patent would result in a pH of less than 7.0 upon its dispersion in saliva and the '866 patent does not provide fentanyl available for oral absorption rather than fast dissolution of fentanyl citrate salt in the saliva. Because of fast dissolution in saliva, fentanyl citrate will be carried down to the GI tract, leading to the alternate mode of bioavailability.

Effervescent technology of administering fentanyl citrate to the buccal cavity has been disclosed in U.S. Pat. Nos. 6,200,604 and 6,974,590; and US Patent Application Nos. 2005/0064030 and 2006/0292219 (Pather et. al.) The disclosures involve a composition of a buccal and/or sublingual tablet using at least one effervescent agent and it is claimed that the effervescent agent (a gas generating agent) is responsible for the transportation of fentanyl across the buccal membranes. The pH of the tablet upon contact with saliva is claimed as substantially neutral, or at a pH slightly higher than 7. Fentanyl citrate buccal tablets formulated by using effervescent technology as disclosed by Pather et al., have been marketed by Cephalon Inc., in the United States under the Brand name Fentora®. Fentora® tablets manufactured using effervescent technology claim that the bioavailability of buccal administration of Fentanyl is enhanced by the carbon dioxide gas generated by the sodium bicarbonate present in the tablet matrix. Such gas reaction happens only at a pH less than 7.0 and essentially requires an acid to liberate the gas from the matrix. The reaction of sodium bicarbonate with an acid (citric acid) in presence of water from the saliva evolves carbon dioxide believed to help the absorption of Fentanyl in the buccal tissues or membranes. Therefore such effervescent tablets are very labile to moisture absorption and lead to poor stability unless packed using special packaging materials. Absorption of moisture triggers the effervescent reaction and leads to instability of the tablet. Therefore preparation of effervescent tablet blends and the handling of these blends is extremely difficult regarding both manufacturing and storage of the products. Similar effervescent technology based Fentanyl administration is also disclosed in US patent application 2005/0142198 (Moe et al).

Bredenberg et al. (Eur. J. Pharma. Sci., 20, 2003, 327-334) disclose dry blending a formulation for rapidly absorbed small sublingual fentanyl tablets. Fentanyl content in these tablets, having a mean weight of approximately 70 mg, is 0.9% for the 400 μg dosage, with a content uniformity of about 88 to 94%. The average uniformity content of fentanyl in these tablets is about 96% for the tablet weight of about 70 mg prepared by direct compression of dry blend of Mannitol with fentanyl citrate of a calculated particle size of about 1 μm (surface area 2.3 m²/g). It was concluded that minor segregation occurs during tablet processing as well.

The use of buffers in oral tablet compositions is also known in the art. The pKa of fentanyl is between about 7.3 and 8.4, and therefore if the pH of the preparation falls below 7.0, it is unlikely that the drug would be readily available for transmucosal absorption. At a pH of about 7.0, the majority of fentanyl molecules are ionized (the nonionized form is more readily absorbed, see, Streisand et al., Anesthesiology 82(3):759-764 (1995)). Eventually at pH higher than 7.3, the absorption of Fentanyl on the transmucosal membrane is favored due to the availability of non-ionized fentanyl around the tablet, which is in contact with the membrane.

The present invention has disclosed the use of inorganic bases such as alkali metal and alkaline earth metal oxides and hydroxides which can convert ionic fentanyl to the unionized fentanyl. Even though the use of basic buffer components to drive the pH to a basic medium to convert the drug into the unionized form to enhance its absorption in a chewing gum or transdermal patch is known, no prior art is known to compose a rapid disintegration buccal tablet suitable to deliver fentanyl without using effervescent agents. The present invention discloses a composition of a direct compression buccal tablet containing the drug, a base, and a disintegrant to administer fentanyl through buccal or sublingual absorption without the use of an effervescent agent. The compositions of the present invention may be useful for variety of drug substances to prepare stable formulations for oral, transmucosal delivery.

### SUMMARY OF THE INVENTION

The addition of a metal oxide and/or a metal hydroxide to an opioid-containing composition facilitates delivery of the drug, such as using magnesium oxide for transbuccal delivery in the mouth to promote a favorable pH for transbuccal administration, and disintegration of an oral dissolvable tablet or lozenge.

The present invention relates to a novel composition of oral transmucosal dosage form to deliver high potency drugs more efficiently and effectively to the buccal and sublingual membranes. The choice of using a base to promote the conversion of ionized fentanyl to unionized fentanyl is disclosed. The novel composition of fentanyl buccal tablet disclosed in this invention contains at least one alkali or alkaline earth metal oxide, or hydroxide which is orally acceptable.

A novel composition of matter disclosed in this invention comprises Fentanyl at about less than 2% (w/w), a filler material such as a carbohydrate at about more than 50% (w/w), a disintegrant at a level of about 0.1% to 12% (w/w), an alkali or alkaline earth metal oxide or hydroxide at a level to get a pH in the range of about 6 to 11, an acid at a level of about less than 10% (w/w), and Lubricant/gliding agent/anticaking agent at a level of about less than 5% (w/w).

An embodiment of the present invention comprises an oral composition for a dosage form comprising an opioid, an acid, at least one disintegrant, and at least one metal oxide and/or metal hydroxide. The metal oxide or metal hydroxide is selected from a group consisting of sodium, lithium, or potassium, or a combination thereof.

The metal oxide or metal hydroxide may also be selected from a group consisting of calcium or magnesium, or a combination thereof.

The dosage form may be selected from a group consisting of a buccal tablet, transmucosal lozenge, sublingual tablet, oral tablet, or rapidly disintegrating tablet, or a combination thereof. The at least one disintegrant may be selected from a group consisting of croscarmellose sodium or starch glycolate, or a combination thereof.

The opioid may be selected from a group consisting of fentanyl, fentanyl citrate, codeine, morphine, hydrocodone, oxycodone, sufentanyl, or remifentanyl, or a combination thereof. The metal oxide is optionally provided in combination with an acid.

The acid may be is selected from a group consisting of citric acid, fumaric acid, maleic acid, tartaric acid, or a combination thereof. The metal oxide may be water insoluble. The metal hydroxide may be water insoluble.

The composition may have no gaseous products form when the composition is in contact with saliva. Furthermore, the composition is micronized for uniform distribution. The dimensions of the dosage form may fit conveniently into an oral cavity.

In another embodiment of the present invention, a composition used to administer low dosage form drugs comprises a pharmaceutical agent, a pharmaceutically acceptable filler, a disintegrant, a lubricant, a buffer, and a non-effervescent base; said pharmaceutical agent having an ionized and unionized form and being micronized for uniform distribution. The base promotes the conversion of the pharmaceutical agent from ionized to unionized form.

The pharmaceutical agent of the composition may be at a level of less than 5 mg per unit dosage form. The pharmaceutical agent may be selected from a group consisting of fentanyl, sufentanyl, remifentanyl, nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, protriptyline, propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, phentolamine, lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, dantrolene, danazol, testosterone cypionate, fluoxymesterone, ethyltestosterone, testosterone enathate, methyltestosterone, fluoxymesterone, testosterone cypionate, estradiol, estropipate, methoxyprogesterone acetate, norethindrone acetate triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, hydrocortisone sodium succinate, and levothyroxine sodium.

The pharmaceutically acceptable filler may be selected from a group consisting of mannitol, gelatin, or lactose. The buffer is anhydrous citric acid. Other buffers known in the art may also be used.

The base may be selected from a group consisting of magnesium oxide, magnesium hydroxide, calcium oxide, calcium hydroxide, tromethamine, lithium hydroxide, lithium oxide, sodium oxide, or sodium hydroxide, or a combination thereof. Other bases known in the art may also be used.

The disintegrant may be selected from a group consisting of croscarmelose sodium or sodium starch glycolate, or a combination thereof. The lubricant may be selected from a group consisting of magnesium stearate or sodium stearylfumarate, or a combination thereof. Other disintegrants known in the art may also be used.

The dosage form may be selected from a group consisting of a buccal tablet, transmucosal lozenge, sublingual tablet, oral tablet, or rapidly disintegrating tablet, caplet, hard capsule, soft capsule, cachet, troche, dissolvable tab, or a combination thereof. Other dosage forms known in the art may also be used

The composition may further comprise additional components selected from the group consisting of surface active agents, dispersing agents, inert diluents, granulating and disintegrating agents, binding agents, lubricating agents, sweetening agents, flavoring agents, coloring agents, preservatives, physiologically degradable agents, aqueous solvents, oily solvents, suspending agents, dispersing agents, wetting agents, demulcents, salts, thickening agents, emulsifying agents, antioxidants, stabilizing agents, and pharmaceutically acceptable polymeric materials.

The total weight of the dosage form may be from about 5 mg to about 250 mg, preferably from about 100 mg to about 175 mg. The base of the composition may be water insoluble. The dimensions of the dosage form may fit conveniently into an oral cavity.

In another embodiment of the present invention, a composition for a drug mixture comprises less than 2% (w/w) fentanyl, more than 50% (w/w) filler material, a disintegrant from about 0.1% (w/w) to about 12% (w/w), an oxide selected from a group consisting of an alkali metal oxide, alkaline earth metal oxide, or hydroxide, less than 10% (w/w) acid, less than 5% (w/w) lubricant, wherein the pH of the drug mixture is from about 6 to about 11. The filler material of the composition may be a carbohydrate.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### DETAILED DESCRIPTION OF THE INVENTION

Fentanyl is a very powerful narcotic analgesic that requires a consistent and uniform dosage form formulation procedure. Therefore, it is worth developing a formulation process for making microgram dosage units of buccal tablets of Fentanyl citrate. For a drug to be suitable for buccal delivery, it is required that the drug substance be significantly absorbed by the buccal tissues. Fentanyl citrate is an almost neutral salt of Fentanyl base and citric acid. Fentanyl citrate in salt form exhibits kinetic equilibrium between the free base and the salt form upon contact with saliva. The absorption of the drug substance by buccal tissues will be maximized when the equilibrium is shifted towards the unionized form.

Oral administration of fentanyl citrate requires a precise and consistent formulation technique to address pharmacologically acceptable uniform drug content among multiple single dosage units and within a single dosage unit. The buccal cavity refers to the space between the cheek and the gum. A buccal tablet administered at the buccal cavity is flanged on one side by the gum tissues and the other side by the cheek tissues or membranes, such as membranes in the mouth. The absorption of a drug, such as fentanyl citrate, in the buccal tissues and membrane begins the moment the drug comes out of the surface of the tablet or on the surface of the tablet itself. Such microscopic absorption of drug can be accelerated by a base (such as when the drug is a base) or an acid (such as when the drug is an acid). Similarly the absorption of the drug can be decelerated by use of use a disintegrant and a buffer in combination with an acid (such as when the drug is a base) or a base (such as when the drug is an acid).

According to one embodiment of this invention, a novel composition of buccal tablet is used to administer low dosage drugs such as fentanyl citrate. The buccal tablet comprises a pharmaceutical agent (drug) at a level of less than 5 mg per unit dosage form, a pharmaceutically acceptable filler, a base typically selected from the group of oxides and hydroxides of alkali/alkaline earth metals, and optionally a buffer system. The base can be either a part of the buffer component or can be different than the buffer used in the composition.

In general low dosage drugs are micronized to establish a uniform distribution of the drug between the individual dosage units as well as within the unit dosage form. Microscopic absorption of fentanyl, according to an embodiment of this invention, is regulated by the usage of micronized buffer and a base.

According to a specific embodiment of the invention, the composition of Fentanyl citrate buccal delivery tablets comprises the components provided in Table-1.

TABLE 1
Novel composition of Fentanyl buccal tablet

| # | Components | Type of Ingredient |
|---|---|---|
| 1 | Fentanyl Citrate - Micronized | Drug |
| 2 | Mannitol | Filler |
| 3 | Croscarmelose sodium | Disintegrant |
| 4 | Magnesium Oxide | Base |
| 5 | Citric Acid | Optional Buffer component |
| 6 | Magnesium Stearate | Lubricant |

One embodiment of the invention provides a technique to achieve better content uniformity between the buccal tablets and a uniform drug distribution within the single dosage form. The acceptable average particle diameter of the drug is of about 1 μm to about 5 μm. The release of fentanyl base from the matrix is mainly controlled by the reaction of fentanyl citrate and magnesium oxide or hydroxide. The released fentanyl base, which is slightly soluble in saliva in the buccal cavity, is readily absorbed through the buccal tissues. Adsorption of fentanyl over the buccal surface is enhanced by magnesium oxide and therefore the absorption rate of fentanyl is faster than disintegration rate of the tablet. This allows for quick pain relief in breakthrough or severe pain management for patients.

Further details of this invention are demonstrated by the examples furnished herein. In these examples, all times, temperatures, and amounts are exact to a certain degree, and also have an error of a certain degree, both as would generally be expected for these types of experiments. In all of the experiments, the fentanyl citrate was micronized by milling, such as with a jet mill, although other methods of milling, and other methods of particle preparation, are suitable and within the scope of this invention. Blending of dry ingredients is accomplished in a V-blender, as in the examples, although other blending methods are contemplated by this invention. Although fentanyl citrate is used as the active ingredient in the examples, it should be appreciated that other active ingredients can be provided for tablets administrable for buccal, sublingual or transmucosal delivery using the present invention.

As is conventional in tabletting a dry composition, an excipient (non-active ingredient) is used as the carrier or filler or matrix material. Other adjuvants, such as disintegrants, glidants, diluents, or lubricants, or a combination thereof, may also be present, as well as the more conventional colorants, flavorings, sweeteners, or other organoleptically-effecting materials, or a combination thereof.

Another embodiment of this invention provides a technique to achieve better drug content uniformity between the dosage forms and a uniform drug distribution within the single dosage form. Preferred drugs used in this invention include, fentanyl, sufentanyl, remifentanyl, antidepressants (e.g., nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, and protriptyline,); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium.

Further details of this invention are shown in the following examples, which should be taken as descriptive and illustrative of aspects of the invention and not limiting the scope of the invention.

Pharmaceutical compositions disclosed in the invention may be prepared, packaged, or sold in formulations suitable for oral administration. The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single- or multi-dose unit.

As used herein, “additional components” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; demulcents; buffers; salts; thickening agents; fillers; emulsifying agents; antioxidants; stabilizing agents; pharmaceutically acceptable polymeric or hydrophobic materials, as well as other components and agents.

Although the descriptions of pharmaceutical compositions provided herein are principally directed to pharmaceutical compositions suitable for administration to humans, it is to be understood by a skilled artisan, based on this disclosure, that such compositions are generally suitable for administration to any mammal. Preparation of compositions suitable for administration to various animals is well understood, and a veterinary pharmacologist of ordinary skill can design and perform such modifications for different animals with routine experimentation based on pharmaceutical compositions for administration to humans.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the drug. The amount of drug in each unit dose is generally equal to the total amount of the drug administered or a convenient fraction of a total dosage amount such as, for example, one-half or one-third of such a dosage.

A formulation of a pharmaceutical composition of the invention suitable for oral administration may be in the form of a discrete solid dosage unit. Solid dosage units include, for example, a tablet, a caplet, a hard or soft capsule, a cachet, a troche, or a dissolvable tab. Each solid dosage unit contains a predetermined amount of the drug, for example a unit dose or fraction thereof. Other formulations suitable for administration include, but are not limited to, a powdered or granular formulation.

A tablet comprising the drug may be made, for example, by compressing or molding the drug, optionally containing one or more additional components. Compressed tablets may be prepared by compressing, in a suitable device, the drug in a free-flowing form such as a powder or granular preparation, and then optionally mixing with one or more of a binder, a lubricant, a glidant, an excipient, a surface active agent and a dispersing agent. Molded tablets may be made by molding in a suitable device, a mixture of the drug, a pharmaceutically acceptable carrier, and at least sufficient liquid to moisten the mixtures.

Tablets may further comprise a sweetening agent, a flavoring agent, a coloring agent, a preservative, or some combination of these in order to provide pharmaceutically elegant and palatable preparations.

Hard capsules comprising the pharmaceutical agent may be made using a physiologically degradable composition, such as gelatin. Such hard capsules comprise the active ingredient, and may further comprise additional components including, for example, an inert solid diluent. Soft gelatin capsules comprising the pharmaceutical agent may also be made using a physiologically degradable composition, such as gelatin. Such soft capsules comprise the pharmaceutical agent, which may be mixed with water or an oil medium.

Powdered and granular formulations according to the invention may be prepared using known methods or methods to be developed. Such formulations may be administered directly to a subject, or used, for example, to form tablets or to fill capsules. Powdered or granular formulations may further comprise one or more of a dispersing or wetting agent, a suspending agent, and a preservative. Additional excipients, such as fillers and sweetening, flavoring, or coloring agents, may also be included in these formulations.

Tablets and pills of the present invention can additionally be prepared with release-controlling coatings. Such a coating may be colored with a pharmaceutically accepted dye. The amount of dye and other excipients in the coating may vary. The coating generally comprises film-forming polymers such as hydroxy-propyl cellulose, hydroxypropylmethyl cellulose, cellulose ester or ether, in acrylic polymer or a mixture of polymers. The coating solution is generally an aqueous solution that may further comprise propylene glycol, sorbitan monooleate, sorbic acid, or fillers such as titanium dioxide, a pharmaceutically acceptable dye.

The solid pharmaceutical compositions of the present invention may further include diluents. Diluents for solid compositions include, for example, microcrystalline cellulose (e.g. AVICEL®), silicified microcrystalline cellulose, microfine cellulose, lactose, starch, pregelatinized starch, calcium sulfate, sugar, dextrates, dextrin, dextrose, dibasic calcium phosphate dihydrate, tribasic calcium phosphate, kaolin, magnesium oxide, maltodextrin, mannitol, dextrates (e. g. EMDEX), hydrated dextrates, polymethacrylates (e.g. Eudragit®), potassium chloride, powdered cellulose, sodium chloride, sorbitol and talc.

Solid pharmaceutical compositions of the present invention may further include binders, e.g.,acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

Solid pharmaceutical compositions of the present invention may further include disintegrants such as alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, polacrilin potassium, powdered cellulose, pregelatinized starch, sodium alginate, sodium starch glycolate (e.g. EXPLOTAB®), hydroxypropylcellulose, methylcellulose, povidone or starch. Glidants, such as, colloidal silicon dioxide, magnesium trisilicate, powdered cellulose, starch, talc and tribasic calcium phosphate may also be added.

Other pharmaceutical additives of the present invention may include: (i) lubricants such as magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate; (ii) flavoring agents and flavor enhancers such as vanillin, ethyl vanillin, menthol, citric acid, fumaric acid ethyl maltitol, and tartaric acid; (iii) pharmaceutically acceptable colorants; (iv) artificial sweeteners such as polyhydric alcohols, e.g., sorbitol, mannitol, xylitol, saccharin, sachharin sodium, aspartame, sucralose and maltitol; and, (v) natural sweeteners, such as glucose, fructose, sucrose and the like.

The dosage unit should have dimensions which fit conveniently into the oral cavity. For example, suitable dimensions for the dosage unit are about 2 mm to about 5 mm in diameter, preferably not exceeding about 7 mm in diameter, and about 0.3 to about 3 mm in thickness, preferably about 0.5 to 3.0 mm in thickness, most preferably about 1.5 to 2.5 mm in thickness. The preferred ratio of the thickness of the tablets to the diameter of the tablets ranges from about 0.1 to about 0.5. The total weight of the dosage unit may be from about 5 mg to about 250 mg, preferably 100 mg to about 175 mg.

In general, low dosage drugs, such as fentanyl, are micronized to effect a uniform distribution of the drug between the individual dosage units as well as within the unit dosage. It may also be important to micronize the absorption accelerator so that the pharmaceutical agent and the absorption accelerator are evenly distributed throughout the matrix. For example, in one embodiment, both fentanyl citrate and the absorption accelerator, magnesium oxide, are micronized. In this embodiment, the average particle diameter is from about 1 μm to about 5 μm. The particular size distribution of the drug and absorption accelerator will be determined through routine experimentation by a pharmaceutical formulator.

In one embodiment, the components of a dosage form such as a tablet include: (i) fentanyl citrate; (ii) mannitol; (iii) citric acid; (iv) croscarmelose sodium; (v) magnesium oxide; (vi) magnesium stearate; and, optionally (viii) an artificial or natural sweetener. The base magnesium oxide may optionally be replaced by magnesium hydroxide, calcium hydroxide, or lithium hydroxide. More preferable disintegrants may include Croscarmelose sodium, sodium starch glycolate and crospovidone. It should be noted that these ranges do not represent absolute numerical values, i.e., the range of fentanyl may be from about 0.1% (w/w) to about 2.0% (w/w), etc. It is well understood by a person of ordinary skill in the art, such as a pharmaceutical formulator, that the percentages in a particular formulation can be adjusted without undue experimentation.

Use of a water insoluble base helps to increase the pH of the medium at a steady rate establishing a dynamic pH gradient against time. Especially by using magnesium oxide, as it is insoluble in water, the magnesium oxide absorbs water to form magnesium hydroxide which in turn increases the pH of the medium.

Also disclosed in this invention is that use of such water insoluble base helps to maintain the pH of the medium irrespective of the volume of the solvent in the range of 1 to 5 mL. Even though the human buccal cavity is small it may vary in size between person to person. Therefore the pH of the tablet in the buccal cavity is the most important term which determines the absorption of fentanyl into the buccal membrane. Water soluble bases give variable pH (more than 1 unit) with volume of solvent between 1 to 5 mL. Thus uniform dosage administration between subjects is very difficult to achieve. However by using water insoluble bases, the pH does not vary significantly with changes in solvent volume between 1 to 5 mL.

In another embodiment of the present invention, the components of a dosage form such as a tablet include: (i) fentanyl citrate; (ii) mannitol; (iii) citric acid; (iv) sodium starch glycolate; (v) magnesium oxide; and (vi) magnesium stearate;

In another embodiment, the components of a dosage form such as a tablet include: (i) fentanyl citrate; (ii) mannitol; (iii) citric acid; (iv) Sodium starch glycolate; (v) magnesium oxide; and (vi) sodium stearyl fumarate

In another embodiment, the components of a dosage form such as a tablet include: (i) fentanyl citrate; (ii) dextrates (EMDEX); (iii) citric acid; (iv) croscarmellose sodium; (v) magnesium hydroxide; and (vi) sodium stearyl fumarate.

In another embodiment, the components of a dosage form such as a tablet include: (i) fentanyl citrate; (ii) lactose and mannitol; (iii) citric acid; (iv) croscarmellose sodium; (v) magnesium oxide; and (vi) sodium stearyl fumarate.

In another embodiment, the components of a dosage form such as a tablet include: (i) fentanyl citrate; (ii) mannitol; (iii) citric acid; (iv) croscarmellose sodium; (v) calcium hydroxide; and (vi) magnesium stearate.

Optionally the composition disclosed in this invention may contain a flavoring agent or a sweetener, or a combination thereof.

In general, the absorbance of basic drugs into the human body is enhanced by the presence of a base. However the stability of the drug in presence of the base can be problematic. Most of the basic drugs that fall under this category are primary, secondary and tertiary amines. The drugs, such as fentanyl, are basically an amine and additionally have an amide function. The stability of the drug in the dosage form is very much dependent on the base used in the composition. Fentanyl is administered at very low dosage level between 100 micrograms to about 1600 micrograms per dose. The quantity of base used and the nature of the base used are the key factors which decide the delivery of the medicaments without compromising the loss of potency due to poor stability. Fentanyl has a propananilide group which is sensitive to a strong base and to hydroxide releasing agents. Though several bases are found suitable for making rapidly disintegrating buccal tablets to administer Fentanyl to the buccal membrane, the stability of the product is very much dependent of the type of base used. The use of hydroxide releasing agents such as sodium hydroxide causes a significant degradation of Fentanyl due to cleavage of the amide function and the formation of N-oxides. Use of an alkaline earth metal oxide, such as magnesium oxide, is useful to achieve the desired pH without degrading the drug. The detailed compositions of rapidly disintegrating buccal tablets of Fentanyl containing magnesium oxide and a disintegrating agent are described in the following examples, which are exemplary, and are not limited to all possible embodiments of the invention.

The various compositions of the ingredients and the range are provided in the following Tables 2 to 7.

TABLE 2
Fentanyl buccal dosage composition

| | Range, % w/w in a Unit |
|---|---|
| Components | dosage |
| Fentanyl Citrate - Micronized | 0.1-2.0 |
| Mannitol | 60 to 97 |
| Croscarmelose sodium | 1 to 6 |
| Magnesium oxide | Less than 2 |
| Citric Acid | 0.1 to 2.0 |
| Magnesium Stearate | 0.3 to 1.5 |
| Sweetener - artificial or natural | Optional |
| Unit dosage form weight range | 100 to 175 mg |

TABLE 3
Fentanyl buccal dosage composition

| | Range, % w/w in a Unit |
|---|---|
| Components | dosage |
| Fentanyl Citrate - Micronized | 0.1-2.0 |
| Mannitol | 60 to 97 |
| Sodium Starch Glycolate | 1 to 12 |
| Magnesium oxide | Less than 2 |
| Citric Acid | 0.1 to 2.0 |
| Magnesium Stearate | 0.3 to 1.5 |
| Sweetener - artificial or natural | Optional |
| Unit dosage form weight range | 100 to 175 mg |

TABLE 4
Fentanyl buccal dosage composition

| | Range, % w/w in a Unit |
|---|---|
| Components | dosage |
| Fentanyl Citrate - Micronized | 0.1-2.0 |
| Mannitol | 60 to 97 |
| Croscarmelose sodium | 1 to 6 |
| Magnesium oxide | Less than 2 |
| Citric Acid | 0.1 to 2.0 |
| Sodium stearyl fumarate | 0.5 to 8 |
| Sweetener - artificial or natural | Optional |
| Unit dosage form weight range | 100 to 175 mg |

TABLE 5
Fentanyl buccal dosage composition

| | Range, % w/w in a Unit |
|---|---|
| Components | dosage |
| Fentanyl Citrate - Micronized | 0.1-2.0 |
| Mannitol | 60 to 97 |
| Sodium Starch Glycolate | 1 to 12 |
| Magnesium oxide | Less than 2 |
| Citric Acid | 0.1 to 2.0 |
| Sodium stearyl fumarate | 0.5 to 8 |
| Sweetener - artificial or natural | Optional |

TABLE 6
Fentanyl buccal dosage composition

| | Range, % w/w in a Unit |
|---|---|
| Components | dosage |
| Fentanyl Citrate - Micronized | 0.1-2.0 |
| Dextrates (EMDEX) | 60 to 97 |
| Sodium Starch Glycolate | 1 to 12 |
| Magnesium oxide | Less than 2 |
| Citric Acid | 0.1 to 2.0 |
| Sodium stearyl fumarate | 0.5 to 8 |
| Sweetener - artificial or natural | Optional |

TABLE 7
Fentanyl buccal dosage composition

| | Range, % w/w in a Unit |
|---|---|
| Components | dosage |
| Fentanyl Citrate - Micronized | 0.1-2.0 |
| Mannitol | 60 to 97 |
| Croscarmellose sodium and | 1 to 12 |
| or Sodium | |
| Starch Glycolate | |
| Calcium hydroxide | Less than 2 |
| Citric Acid | 0.1 to 2.0 |
| Magnesium stearate | 0.5 to 8 |
| Sweetener - artificial or natural | Optional |

The invention of this disclosure is additionally demonstrated by the following examples. Combination or deletions of an ingredient between the listed examples are within the scope of this invention.

### EXAMPLE 1

TABLE 8
Composition of 100 microgram Fentanyl buccal tablet

| | | | | |
|---|---|---|---|---|
| 1 | Mannitol (Pearlitol | Filler | mg | 107.530 |
| | 100 SD) | | | |
| 2 | Citric acid anhydrous | buffer | mg | 1.000 |
| 3 | Croscarmelose sodium | Disintegrant | mg | 3.750 |
| 4 | Magnesium Oxide | Base | mg | 0.558 |
| 5 | Fentanyl Citrate - | Drug | mg | 0.157 |
| | Micronized | | | |
| 6 | Magnesium Stearate | Lubricant | mg | 2.000 |
| | Tablet weight, mg | | | 115.00 |

### EXAMPLE 2

TABLE 9
Composition of 200 microgram Fentanyl buccal tablet

| | | | | |
|---|---|---|---|---|
| 1 | Mannitol (Pearlitol | Filler | mg | 107.373 |
| | 100 SD) | | | |
| 2 | Citric acid anhydrous | buffer | mg | 1.000 |
| 3 | Croscarmelose sodium | Disintegrant | mg | 3.750 |
| 4 | Magnesium Oxide | Base | mg | 0.558 |
| 5 | Fentanyl Citrate - | Drug | mg | 0.314 |
| | Micronized | | | |
| 6 | Magnesium Stearate | Lubricant | mg | 2.000 |
| | Tablet weight, mg | | | 115.00 |

### EXAMPLE 3

TABLE 10
Composition of 400 microgram Fentanyl buccal tablet

| | | | | |
|---|---|---|---|---|
| 1 | Mannitol (Pearlitol 100 SD) | Filler | mg | 107.059 |
| 2 | Citric acid anhydrous | buffer | mg | 1.000 |
| 3 | Croscarmelose sodium | Disintegrant | mg | 3.750 |
| 4 | Magnesium Oxide | Base | mg | 0.558 |
| 5 | Fentanyl Citrate - | Drug | mg | 0.628 |
| | Micronized | | | |
| 6 | Magnesium Stearate | Lubricant | mg | 2.000 |

| | |
|---|---|
| Tablet weight, mg | 115.00 |

### EXAMPLE 4

TABLE 11
Composition of 800 microgram Fentanyl buccal tablet

| | | | | |
|---|---|---|---|---|
| 1 | Mannitol (Pearlitol 100 SD) | Filler | mg | 106.431 |
| 2 | Citric acid anhydrous | buffer | mg | 1.000 |
| 3 | Croscarmelose sodium | Disintegrant | mg | 3.750 |
| 4 | Magnesium Oxide | Base | mg | 0.558 |
| 5 | Fentanyl Citrate - | Drug | mg | 1.256 |
| | Micronized | | | |
| 6 | Magnesium Stearate | Lubricant | mg | 2.000 |

| | |
|---|---|
| Tablet weight, mg | 115.00 |

### EXAMPLE 5

TABLE 12
Composition of 400 microgram Fentanyl buccal tablet

| | | | | |
|---|---|---|---|---|
| 1 | Mannitol (Pearlitol 100 SD) | Filler | mg | 110.069 |
| 2 | Citric acid anhydrous | buffer | mg | 1.000 |
| 3 | Croscarmelose sodium | Disintegrant | mg | 4.500 |
| 4 | Magnesium hydroxide | Base | mg | 1.800 |
| 5 | Fentanyl Citrate - | Drug | mg | 0.628 |
| | Micronized | | | |
| 6 | Magnesium Stearate | Lubricant | mg | 2.000 |

| | |
|---|---|
| Tablet weight, mg | 120.00 |

### EXAMPLE 6

TABLE 13
Composition of 100 microgram Fentanyl buccal tablet

| | | | | |
|---|---|---|---|---|
| 1 | Mannitol (Pearlitol 100 SD) | Filler | mg | 71.540 |
| 2 | Citric acid anhydrous | buffer | mg | 1.000 |
| 3 | Lactose | Filler | mg | 39.000 |
| 4 | Croscarmelose sodium | Disintegrant | mg | 4.500 |
| 5 | Magnesium hydroxide | Base | mg | 1.800 |
| 6 | Fentanyl Citrate - | Drug | mg | 0.157 |
| | Micronized | | | |
| 7 | Magnesium Stearate | Lubricant | mg | 2.000 |

| | |
|---|---|
| Tablet weight, mg | 120.00 |

### EXAMPLE 7

TABLE 14
Composition of 100 microgram Fentanyl buccal tablet

| | | | | |
|---|---|---|---|---|
| 1 | Mannitol (Pearlitol 100 SD) | Filler | mg | 111.840 |
| 2 | Citric acid anhydrous | buffer | mg | 1.000 |
| 3 | Croscarmelose sodium | Disintegrant | mg | 4.500 |
| 4 | Calcium Hydroxide | Base | mg | 0.500 |
| 5 | Fentanyl Citrate - | Drug | mg | 0.157 |
| | Micronized | | | |
| 6 | Magnesium Stearate | Lubricant | mg | 2.000 |

| | |
|---|---|
| Tablet weight, mg | 120.00 |

### EXAMPLE 8

TABLE 15
Composition of 800 microgram Fentanyl buccal tablet

| | | | | |
|---|---|---|---|---|
| 1 | Mannitol (Pearlitol 100 SD) | Filler | mg | 110.741 |
| 2 | Citric acid anhydrous | buffer | mg | 1.000 |
| 3 | Croscarmelose sodium | Disintegrant | mg | 4.500 |
| 4 | Calcium Hydroxide | Base | mg | 0.500 |
| 5 | Fentanyl Citrate - | Drug | mg | 1.256 |
| | Micronized | | | |
| 6 | Magnesium Stearate | Lubricant | mg | 2.000 |

| | |
|---|---|
| Tablet weight, mg | 120.00 |

### EXAMPLE 9

TABLE 16
Composition of 2000 microgram Fentanyl buccal tablet

| | | | | |
|---|---|---|---|---|
| 1 | Mannitol (Pearlitol 100 SD) | Filler | mg | 108.857 |
| 2 | Citric acid anhydrous | buffer | mg | 1.000 |
| 3 | Croscarmelose sodium | Disintegrant | mg | 4.500 |
| 4 | Calcium Hydroxide | Base | mg | 0.500 |
| 5 | Fentanyl Citrate - | Drug | mg | 3.140 |
| | Micronized | | | |
| 6 | Magnesium Stearate | Lubricant | mg | 2.000 |

| | |
|---|---|
| Tablet weight, mg | 120.00 |

### EXAMPLE 10

TABLE 17
Composition of 400 microgram Fentanyl buccal tablet

| | | | | |
|---|---|---|---|---|
| 1 | Mannitol (Pearlitol 100 SD) | Filler | mg | 104.389 |
| 2 | Sodium starch Glycolate | Disintegrant | mg | 12.000 |
| 3 | Citric acid anhydrous | buffer | mg | 1.000 |
| 4 | Magnesium Oxide | Base | mg | 0.483 |
| 5 | Fentanyl Citrate - | Drug | mg | 0.628 |
| | Micronized | | | |
| 6 | Magnesium Stearate(dried) | Lubricant | mg | 1.500 |

| | |
|---|---|
| Tablet weight, mg | 120.00 |

### EXAMPLE 11

TABLE 18
Composition of 400 microgram Fentanyl buccal tablet

| | | | | |
|---|---|---|---|---|
| 1 | Mannitol (Pearlitol 100 SD) | Filler | mg | 100.909 |
| 2 | Sodium starch Glycolate | Disintegrant | mg | 12.000 |
| 3 | Citric acid anhydrous | buffer | mg | 1.000 |
| 4 | PRUV (Sodium | Lube | mg | 5.000 |
| | stearylfumarate) | | | |
| 5 | Fentanyl Citrate - | Drug | mg | 0.628 |
| | Micronized | | | |
| 6 | Magnesium Oxide | Base | mg | 0.458 |

| | |
|---|---|
| Tablet weight, mg | 120.00 |

### EXAMPLE 12

TABLE 19
Composition of 400 microgram Fentanyl buccal tablet

| | | | | |
|---|---|---|---|---|
| 1 | Mannitol (Pearlitol 100 SD) | Filler | mg | 109.349 |
| 2 | Croscarmelose sodium | Disintegrant | mg | 3.500 |
| 3 | Citric acid anhydrous | buffer | mg | 1.000 |
| 4 | PRUV (Sodium | Lube | mg | 5.000 |
| | stearylfumarate) | | | |
| 5 | Fentanyl Citrate - | Drug | mg | 0.628 |
| | Micronized | | | |
| 6 | Magnesium Oxide | Base | mg | 0.523 |

| | |
|---|---|
| Tablet weight, mg | 120.00 |

### EXAMPLE 13

TABLE 20
Composition of 100 microgram Fentanyl buccal tablet

| | | | | |
|---|---|---|---|---|
| 1 | Mannitol (Pearlitol 100 SD) | Filler | mg | 105.943 |
| 2 | Croscarmelose sodium | Disintegrant | mg | 3.500 |
| 3 | Citric acid anhydrous | buffer | mg | 0.400 |
| 4 | PRUV (Sodium | Lube | mg | 5.000 |
| | stearylfumarate) | | | |
| 5 | Fentanyl Citrate - | Drug | mg | 0.157 |
| | Micronized | | | |
| 6 | Tromethamine | Base | mg | 5.000 |

| | |
|---|---|
| Tablet weight, mg | 120.00 |

### EXAMPLE 14

TABLE 21
Composition of 100 microgram Fentanyl buccal tablet

| | | | | |
|---|---|---|---|---|
| 1 | Mannitol (Pearlitol 100 SD) | Filler | mg | 111.943 |
| 2 | Citric acid anhydrous | buffer | mg | 1.000 |
| 3 | Croscarmelose sodium | Disintegrant | mg | 4.500 |
| 4 | Lithium Hydroxide | Base | mg | 0.400 |
| 5 | Fentanyl Citrate - | Drug | mg | 0.157 |
| | Micronized | | | |
| 6 | Magnesium Stearate | Lubricant | mg | 2.000 |

| | |
|---|---|
| Tablet weight, mg | 120.00 |

### EXAMPLE 15

TABLE 22
Composition of 100 microgram Fentanyl buccal tablet

| | | | | |
|---|---|---|---|---|
| 1 | Mannitol (Pearlitol 100 SD) | Filler | mg | 111.943 |
| 2 | Citric acid anhydrous | buffer | mg | 1.000 |
| 3 | Croscarmelose sodium | Disintegrant | mg | 4.500 |
| 4 | Sodium Hydroxide | Base | mg | 0.400 |
| 5 | Fentanyl Citrate - | Drug | mg | 0.157 |
| | Micronized | | | |
| 6 | Magnesium Stearate | Lubricant | mg | 2.000 |

| | |
|---|---|
| Tablet weight, mg | 120.00 |

### EXAMPLE 16

TABLE 23
Composition of 100 microgram Fentanyl buccal tablet

| | | | | |
|---|---|---|---|---|
| 1 | Mannitol (Pearlitol 100 SD) | Filler | mg | 110.943 |
| 2 | Citric acid anhydrous | buffer | mg | 1.000 |
| 3 | Croscarmelose sodium | Disintegrant | mg | 4.500 |
| 4 | Magnesium Hydroxide | Base | mg | 1.400 |
| 5 | Fentanyl Citrate - | Drug | mg | 0.157 |
| | Micronized | | | |
| 6 | Magnesium Stearate | Lubricant | mg | 2.000 |

| | |
|---|---|
| Tablet weight, mg | 120.00 |

### EXAMPLE 17

### Procedure for Making Fentanyl Buccal Tablet

The ingredients other than the lubricant were added into V-blender (Patterson-Kelley V-blender, with intensifier bars) and blended for about 15 minutes. After allowing the powder to settle, the lubricant was added and further blended for about 3 minutes. The blend was loaded into a 10 station table press (Ayush Minipress-II, with Natoli punches and 0.2969 inch die) and the feed was adjusted to the desired tablet weight and thickness. The tablets were punched and tested for pH, hardness, disintegration, and related impurities. The tablets were packed and loaded for a stress study at 50° C.

The test results of Fentanyl buccal tablet prepared by the formula for the 100 microgram strength are given in Table-24.

TABLE 24
Choice of Base and its effect on stability of Fentanyl

| | | | | Related | |
|---|---|---|---|---|---|
| | | Related | | impurities, % | |
| | | impurities, | | after 2 Weeks at | |
| | | %, Initial | | 50° C. | |
| Example # | Base Used | N-Oxide | Total | N-Oxide | Total |
| 13 | Tromethamine | 0.01 | 0.1 | | |
| 14 | Lithium hydroxide | 0.01 | 0.1 | | |
| 15 | Sodium hydroxide | 0.01 | 0.1 | | |
| 7 | Calcium hydroxide | 0.01 | 0.1 | 0.07 | 0.2 |
| 16 | Magnesium | 0.01 | 0.1 | 0.05 | 0.2 |
| | hydroxide | | | | |
| 1 | Magnesium oxide | 0.01 | 0.1 | 0.02 | 0.2 |

### EXAMPLE 18

The pH of the tablet prepared in Example 1 was measured in various volumes of water. The results are given in Table-25. A comparison of pH of Fentora 100 μg tablet also is provided.

TABLE 25
pH against Volume:

| # | Water Volume | Tablet prepared in example 1 | Fentora, 100 μg |
|---|---|---|---|
| 1 | 5 | 9.4 | 7.0 |
| 2 | 3 | 9.5 | 7.3 |
| 3 | 2 | 9.5 | 8.1 |
| 4 | 1 | 9.6 | 8.6 |

While the invention has been specifically described in connection with certain specific embodiments thereof, it is to be understood that this is by way of illustration and not of limitation and that various changes and modifications in form and details can be made thereto, and the scope of the appended claims should be construed as broadly as the prior art will permit.

The description of the invention is merely exemplary in nature, and thus, variations that do not depart from the gist of the invention are intended to be within the scope of the invention. Such variations are not to be regarded as a departure from the spirit and scope of the invention.

What is claimed is:

1. A composition comprising an opioid, an acid, at least one disintegrant, and at least one metal oxide or metal hydroxide.

2. The composition of claim 1, wherein the metal is an alkali metal.

3. The composition of claim 2, wherein the alkali metal is selected from a group consisting of sodium, lithium, or potassium, or a combination thereof.

4. The composition of claim 1, wherein the metal is an alkaline earth metal.

5. The composition of claim 4, wherein the alkali earth metal is selected from a group consisting of calcium or magnesium, or a combination thereof.

6. The composition of claim 1, wherein said composition is set in a dosage form, said dosage form.

7. The composition of claim 6,wherein the dosage form is selected from a group consisting of a buccal tablet, transmucosal lozenge, sublingual tablet, oral tablet, rapidly disintegrating tablet, caplet, hard capsule, soft capsule, cachet, troche, or dissolvable tab.

8. The composition of claim 1, wherein the at least one disintegrant is selected from a group consisting of croscarmellose sodium glycolate, or starch glycolate, or a combination thereof.

9. The composition of claim 1, wherein the opioid is selected from a group consisting of fentanyl, fentanyl citrate, codeine, morphine, hydrocodone, oxycodone, sufentanyl, or remifentanyl, or a combination thereof.

10. The composition of claim 1, wherein the metal oxide is provided in combination with an acid.

11. The composition form of claim 1, wherein the acid is selected from a group consisting of citric acid, fumaric acid, maleic acid, tartaric acid, or a combination thereof.

12. The composition of claim 1, wherein the metal oxide is water insoluble.

13. The composition of claim 1, wherein the metal hydroxide is water insoluble.

14. The composition of claim 1, wherein no gaseous products are generated upon contact with saliva.

15. The composition of claim 1, wherein the dosage form is micronized for uniform distribution.

16. A composition used to administer low dosage form drugs comprising:
a pharmaceutical agent, a pharmaceutically acceptable filler, a disintegrant, a lubricant, a buffer, and a non-effervescent base;
said pharmaceutical agent having an ionized and unionized form and being micronized for uniform distribution.

17. The composition of claim 16, wherein the pharmaceutical agent is at a level of less than 5 mg per unit dosage form.

18. The composition of claim 16, wherein the pharmaceutical agent is selected from a group consisting of fentanyl, sufentanyl, remifentanyl, nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, protriptyline, propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, phentolamine, lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, dantrolene, danazol, testosterone cypionate, fluoxymesterone, ethyltestosterone, testosterone enathate, methyltestosterone, fluoxymesterone, testosterone cypionate, estradiol, estropipate, methoxyprogesterone acetate, norethindrone acetate triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, hydrocortisone sodium succinate, and levothyroxine sodium.

19. The composition of claim 16, wherein the pharmaceutically acceptable filler is selected from a group consisting of mannitol, gelatin, or lactose.

20. The composition of claim 16, wherein the buffer is anhydrous citric acid.

21. The composition of claim 16, wherein the base is selected from a group consisting of magnesium oxide, magnesium hydroxide, calcium oxide, calcium hydroxide, tromethamine, lithium hydroxide, lithium oxide, sodium oxide, sodium hydroxide, or a combination thereof.

22. The composition of claim 16, wherein the disintegrant is selected from a group consisting of croscarmelose sodium or sodium starch glycolate, or a combination thereof.

23. The composition of claim 16, wherein the lubricant is selected from a group consisting of magnesium stearate or sodium stearylfumarate, or a combination thereof.

24. The composition of claim 16, wherein the dosage form is selected from a group consisting of a buccal tablet, transmucosal lozenge, sublingual tablet, oral tablet, rapidly disintegrating tablet, caplet, hard capsule, soft capsule, cachet, troche, or dissolvable tab.

25. The composition of claim 16, further comprising additional components selected from the group consisting of surface active agents, dispersing agents, inert diluents, granulating and disintegrating agents, binding agents, lubricating agents, sweetening agents, flavoring agents, coloring agents, preservatives, physiologically degradable agents, aqueous solvents, oily solvents, suspending agents, dispersing agents, wetting agents, demulcents, salts, thickening agents, emulsifying agents, antioxidants, stabilizing agents, and pharmaceutically acceptable polymeric materials.

26. The composition of claim 16, wherein the total weight of the dosage form is from about 5 mg to about 250 mg.

27. The composition of claim 16, wherein the total weight of the dosage form is from about 100 mg to about 175 mg.

28. The composition of claim 16, wherein the base is water insoluble.

29. A composition for a drug mixture comprising:
less than 2% (w/w) fentanyl;
more than 50% (w/w) filler material;
a disintegrant from about 0.1% to about 12% (w/w);
a metal selected from a group consisting of an alkali metal or alkaline earth metal oxide or hydroxide;
less than 10% (w/w) acid; and
less than 5% (w/w) lubricant, wherein the pH of the drug mixture is from about 6 to about 11.

30. The composition of claim 29, wherein the filler material is a carbohydrate.
